(19) United States
(12) Patent Application Publication
(10) Pub. No.: US 2020/0126642 A1
(43) Pub. Date: Apr. 23, 2020

(54) COGNITIVE SOLUTIONS FOR DETECTION OF, AND OPTIMIZATION BASED ON, COHORTS, ARMS, AND PHASES IN CLINICAL TRIALS

(71) Applicant: INTERNATIONAL BUSINESS MACHINES CORPORATION, Armonk, NY (US)

(72) Inventors: Kimberly D. Kenna, Cary, NC (US); Eric Will, Rochester, MN (US); Adam Clark, Rochester, MN (US); David Ranum, Rochester, MN (US)

(21) Appl. No.: 16/165,612

(22) Filed: Oct. 19, 2018

(51) Int. Cl. G16H 10/20; G16H 10/60; G16H 15/00

(52) U.S. Cl. CPC G16H 10/20; G16H 15/00; G16H 10/60

(57) ABSTRACT

Cognitive solutions are provided for detection of, and optimization based on, cohorts, arms, and phases in clinical trials. In various embodiments, a first datastore is accessed. The first datastore comprises a clinical trial description. The clinical trial description includes a reference to a cohort, arm, or phase of a clinical study. The clinical trial description is analyzed to identify an entity type. The entity type is a cohort, arm or phase reference. A clinical attribute associated with the entity type is determined within the clinical trial description. A screening criterion associated with the clinical attribute is determined. A second datastore is accessed. The second datastore comprises a medical record of a patient. The medical record is screened against the screening criterion.

### BACKGROUND

Embodiments of the present disclosure relate to optimization of clinical trials, and more specifically, to cognitive solutions for detection of, and optimization based on, cohorts, arms, and phases in clinical trials.

### BRIEF SUMMARY

According to embodiments of the present disclosure, methods of and computer program products for detecting cohorts, arms and phases in clinical studies are provided. A first datastore is accessed. The first datastore comprises a clinical trial description. The clinical trial description includes a reference to a cohort, arm, or phase of a clinical study. The clinical trial description is analyzed to identify an entity type. The entity type is a cohort, arm or phase reference. A clinical attribute associated with the entity type is determined within the clinical trial description. A screening criterion associated with the clinical attribute is determined. A second datastore is accessed. The second datastore comprises a medical record of a patient. The medical record is screened against the screening criterion.

### BRIEF DESCRIPTION OF THE SEVERAL VIEWS OF THE DRAWINGS

FIG. 1 illustrates an exemplary process for detecting cohorts, arms and phases in clinical studies according to embodiments of the present disclosure.

FIG. 2 is an exemplary unprocessed trial description according to embodiments of the present disclosure.

FIG. 3 is an exemplary processed trial description according to embodiments of the present disclosure.

FIG. 4 is an exemplary processed trial description according to embodiments of the present disclosure.

FIG. 5 is an exemplary processed trial description according to embodiments of the present disclosure.

FIG. 6 illustrates a method for detecting cohorts, arms and phases in a clinical study according to an embodiment of the present disclosure.

FIG. 7 depicts a computing node according to an embodiment of the present disclosure.

### DETAILED DESCRIPTION

Finding clinical trials for patients is challenging and time consuming, especially considering the large quantity of unstructured patient data, an abundance of available trials, and complex protocol criteria to consider. Clinicians have no easy way to search across eligibility criteria of relevant clinical trials for their patients. Similarly, clinical trial offices are not enabled to efficiently identify potential patients for their trials. These inefficiencies remain a barrier to increasing enrollment rates and can hinder research advancement and patient care.

Clinical trial recruitment software systems that assist with matching patients with trials may leverage a query-based approach against a very basic relevancy match of the clinical trial's characteristics. Such an approach lacks any knowledge of the cohorts that exist in the trial. Accordingly, such systems do not contain a deep normalized understanding of the mapping of the cohorts, arms, and phases of the trial to the applicable eligibility criteria.

To the extent that some basic knowledge of cohorts is present, manual curation by an administrator of the system is cost-prohibitive in view of the increasing volume of new studies (and updates to studies) being created every day. For example, in 2005 there were only approximately 12,000 studies in posted on ct.gov. In 2010, that number reached 83,000. By 2017, that number again increased to 263,000.

To address these and other shortcomings of alternative approaches, the present disclosure provides cognitive clinical trial solutions to detect cohorts, arms, and phases, and to use these within a study to enable clinical research efficiencies.

In various embodiments, a clinical trial system is provided that applies cognitive capabilities, including Natural Language Processing (NLP) and Machine Learning (ML) along with specialized aggregation and normalization techniques to automatically discover the unique cohorts, arms, and phases described within a study text.

In various embodiments, the uniquely discovered cohorts, arms, and phases are mapped to appropriate study eligibility criteria using techniques that include the NLP and normalization discussed above as well as association techniques such as anaphora and structural inheritance.

In various embodiments, the same NLP, ML, aggregation, and normalization techniques are applied to cohort/arm/phase information stored as text in an external system to associate the knowledge that the external system has about those cohorts/arms/phases to the detection above. As an example, this may be used to automatically map the status of those cohorts, arms and phases from a source Clinical Trial Management System to associated criteria.

Accordingly, automated cognitive approaches are provided for understanding the cohorts and arms described in natural language within clinical trial documents and related systems. This understanding is applied in various embodiments to a recruitment system. As a result, clinicians may be shown only those trials for which a patient belongs to an actively recruiting cohort. Trial coordinators are able to see which patients in the overall recruitment pipeline match to which cohorts of their study (and the status of those cohorts), so that they can prioritize their patient follow-up work. Trial coordinators may be shown a view that focuses them only on the criteria for the currently open and applicable cohort of the study relevant for a specific patient they are screening and arms/phases of the study that are actively open and hides all the other non-applicable information so that they can efficiently spend their time only on the criteria that matter.

Accordingly, systems and method provided herein enable clinicians to more easily and quickly find a list of clinical trials for an eligible patient, and in the clinical trial office, find patients that are potentially eligible for any of the site's trials. The improvement in screening efficiency and more effective patient recruitment can help increase clinical trial enrollment targets and opportunities to offer patients the option of a clinical trial for treatment.

As used herein, cohort refers to a group of patients that share a set of characteristics.

As used herein, arm refers to a treatment path for a set of patients on a study.

As used herein, phase refers to a progressive iteration level within a group of related studies (typically Phase I-IV).

Multi-cohort studies are becoming more common with the dawn of personalized medicine and multi-arm studies are already a common practice, especially in Phase III trials. In the case of multi-cohort and multi-arm studies, a site that is running the study is often only recruiting patients to a subset of the cohorts and/or arms at a given point in time. The criteria that a patient must meet to enroll into that clinical trial can vary (sometimes greatly) depending on which cohort that patient matches and based on which arm (or treatment path) of the study the patient will go onto once enrolled. Recruitment systems without a knowledge of open cohorts, arms, and phases, put the burden on their end users: the clinicians, trial coordinators, and even patients who must comb through the large result set. These users must figure out if each result in the result set is a match at the more granular level of the cohorts, arms, and phases that are currently open for recruitment at that site. This wastes precious appointment time for the hospital staff, looking at matches for a patient that turn out not to be open for recruitment at that point in time.

When it turns out there is a successful match to an open trial, the user must also then manually read each criterion and determine if that criterion is applicable to the cohort for the patient they are evaluating and if that criteria is applicable to the study arm and phase currently open for enrollment. Reading through criteria that are inappropriate for a given situation again wastes precious time for the clinical research coordinators. This challenge contributes to the up to 80% of sites failing to meet their accrual target timeline goals.

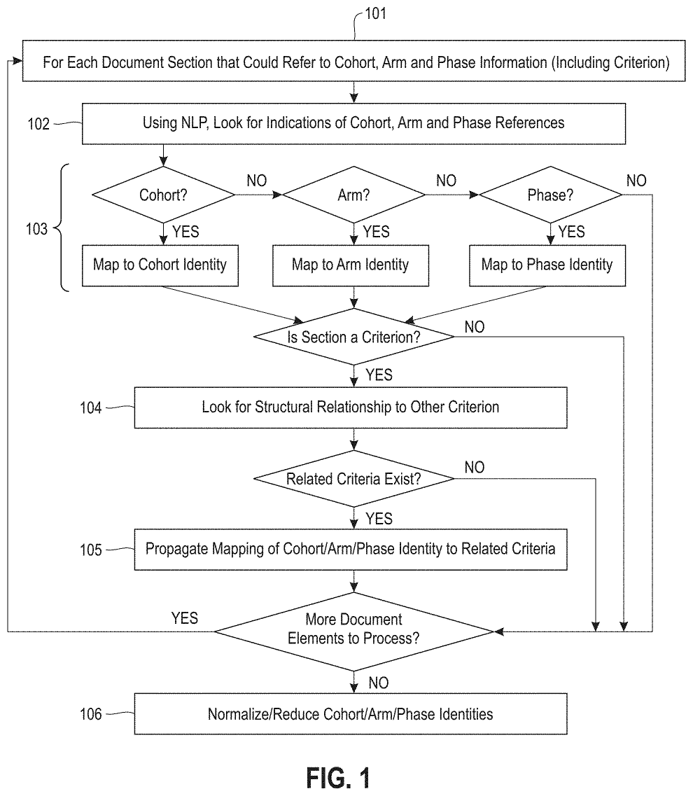

With reference now to FIG. 1, an exemplary process for detecting cohorts, arms and phases in clinical studies is illustrated according to embodiments of the present disclosure.

At 101, eligibility criteria and relevant clinical trial sections are obtained from a clinical trial. Each section is subsequently evaluated as set out below. In particular, for each input document section that could refer to cohort, arm, and phase information, the following steps are performed. It will be appreciated that a variety or structured and unstructured formats are suitable for use as described herein. For example, the XML Schema for ClinicalTrials.gov public XML (available at https://clinicaltrials.gov/ct2/html/images/info/public.xsd; version 2018.08.22 as of this writing) may be used to describe a given clinical trial. Irrespective of whether a given input format provides a hierarchy of separate eligibility criteria, that format may be extended as set out herein.

At 102,using NLP techniques, each section is inspected for indications that it relates to a cohort, arm, or phase. These techniques include, but are not limited to, using NLP to detect concepts that imply a cohort, arm, or phase type is being referenced, inspecting section metadata for indications that the section belongs to a group or group type, and using NLP to an expanded definition of a unique cohort, arm, or phase. For example, disease characteristics associated with a named cohort may be used later in abbreviated form to reference the unique cohort.

At 103, using the result of the techniques described in step 102, the detected entity type (cohort/arm/phase) is mapped to a definition of that type (e.g., HER2-breast cancer cohort) and the section where the entity was detected.

If the section constitutes a criterion of the study, once identification is complete, the intra-document structural relationships are evaluated at 104. Any criteria that should inherit characteristics of other criteria are updated at 105 to include cohort detection. For example, a criterion reading, “all breast cancer patients must be BRCA positive and one of the following,” would be identified as part of the Breast Cancer cohort, but in this step that knowledge would be propagated to any nested child criteria that exist underneath it. The child criteria may not have any indication of being specific to one cohort on their own, but by leveraging the structural relationship, the system can properly annotate these child-criteria as well.

The above process is repeated for all document elements until all criteria are identified as being cohort/arm/phase-specific or not. Once all document elements have been processed, at 106, the cohort/arm/phase entity names within the document are normalized. A variety of techniques may be used for normalization.

Some criteria may explicitly indicate a cohort, arm or phase name (e.g., “Cohort #1”), which can be extracted and understood as explicit using standard NLP techniques. These explicit names are not modified or merged with other explicit names.

Some criteria may include language referencing patient characteristics that must be met for a criterion to apply (e.g., “Breast cancer patients must be BRCA positive”). In this scenario, the antecedent is identified as a potential cohort group name.

Anaphora may be used to identify a cohort, arm or phase name based on prior trial information. For example, if one criterion reads “patients must have Stage 4 breast cancer or Stage 4 lung cancer,” and a subsequent criterion reads, “patients in the first group must be BRCA positive,” the implication is that “the first group” refers to “Stage 4 breast cancer” patients.

Once the above techniques are used to identify the most likely group names, clustering techniques can be used to further reduce redundancy across cohort, arm & phase names. If similar names exist recognizing when two groups overlap in content (e.g., “Breast Cancer Cohort” and “Stage 4 Breast Cancer Cohort”), the system decides which to keep based on configuration and an understanding of which name contains the other. Similarly, two names which were semantically identical (e.g., “Cohort 2” vs “Cohort II”) would be reduced to a single cohort name. Once name normalization completes, the process completes, having identified which criteria belong to cohorts, arms, and phases, and labeled each with the correct corresponding normalized name.

In various embodiments, a recruitment system is provided that incorporates the process described above. This significantly reduces the manual burden placed on end users without requiring manual curation of the trials with this knowledge.

Three use cases are described below, illustrating the recruitment process with and without the above-described processes. Each example is given in terms of a real trial from clinicaltrials.gov: https://clinicaltrials.gov/ct2/show/NCT02791334.

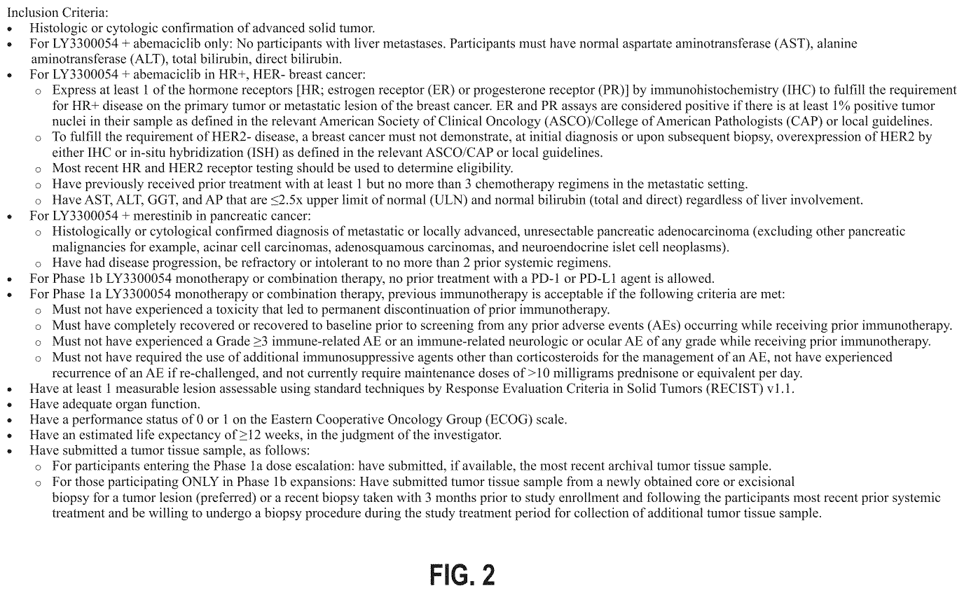

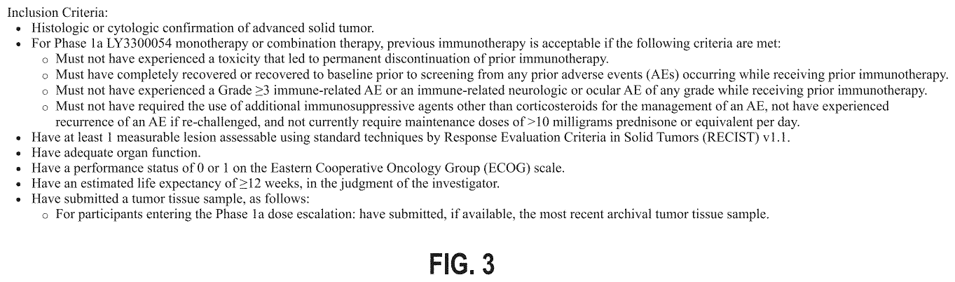

Referring to FIGS. 2-3, a first use case is illustrated. In this exemplary trial the criteria include: a metastatic lung cancer patient (cohort) during phase 1a (phase) of the trial, who has had prior treatment with a PD-L1 agent when the current treatment open is LY3300054+Abemaciclib with dose escalation (arm).

FIG. 2 shows how the exemplary trial would appear in a result set. The user would be asked to assess a candidate patient against all of the listed inclusion criteria.

FIG. 3 shows a reduced set of inclusion criteria according to embodiments of the present disclosure. In particular, after application of the techniques described above, a trial appears in a result set, but the user is shown for evaluation only the subset of inclusion criteria applicable to this patient and the open phase/arm.

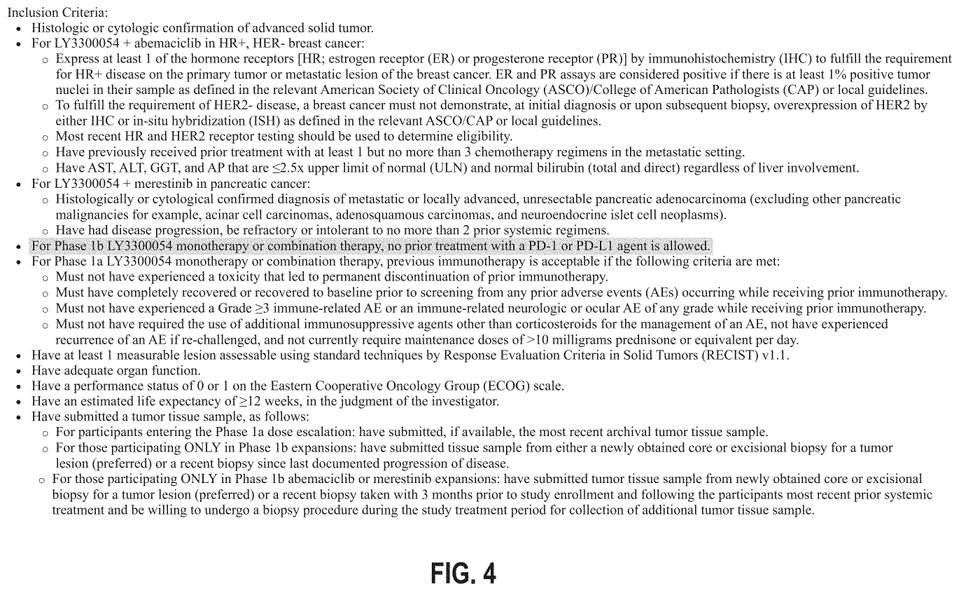

Referring to FIG. 4, a second use case is illustrated. In this exemplary trial the criteria include: a metastatic lung cancer patient (cohort) during phase 1b (phase) of the trial, who has had prior treatment with a PD-L1 agent when the current treatment open is LY3300054+Abemaciclib (arm). In this example, phase 1b of the trial instead of phase 1a.

Without processing of the criteria as set forth above, the user of the recruitment system would consider this patient to be a potential match to the study. Only after manually evaluating the inclusion criteria from top to bottom, reaching about midway through the criteria evaluation (highlighted), would the user realize that this patient is not a fit for the trial because the trial is in phase 1b, and the patient's prior treatment with a PD-L1 agent disqualifies them in this phase.

Application of the processing described herein enables automatic elimination of this patient as a currently viable match for this trial based on the patient data and the open phase of the study.

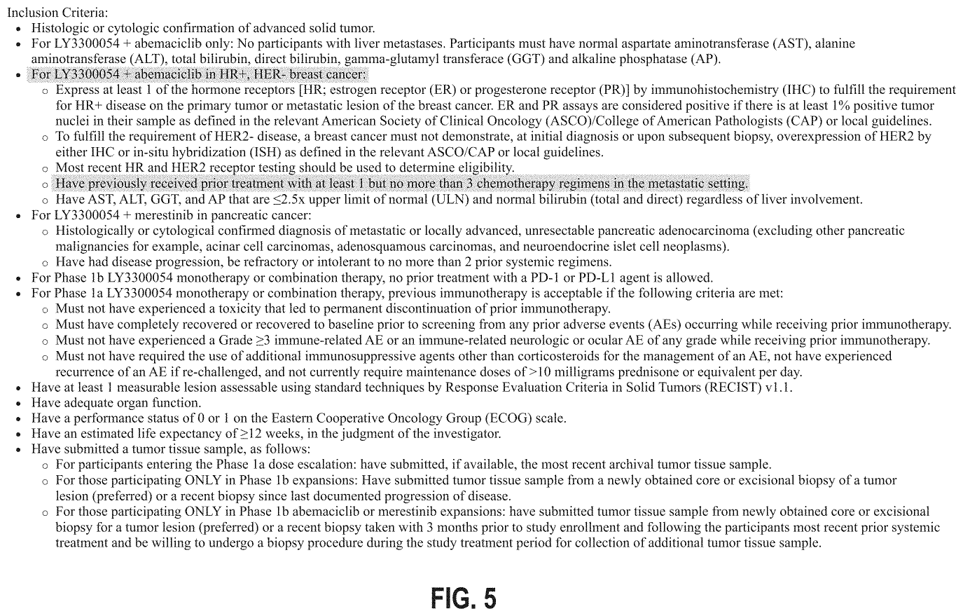

Referring to FIG. 5, a third use case is illustrated. In this exemplary trial the criteria include: an ER+, HER2-breast cancer (cohort) patient that is metastatic to the bone who has had no prior chemotherapy treatment in the metastatic trial is matched during phase 1a (phase) of the trial when LY3300054 + abemaciclib (arm) is the currently open arm.

Without processing of the criteria as set forth above, the trial would appear in the result set and the user would be asked to assess the patient against all the inclusion criteria. Only after reaching the assessment relating to the breast cancer cohort to which the patient is associated, and only in view of the currently open arm, would the user realize that the patient is not a match because they have not had prior chemo yet in the metastatic setting. This is a requirement for this combination of cohort/arm.

Application of the processing described herein enables automatic elimination of this patient as a currently viable match for this trial based on the patient data and the open arm of the study against the criteria as written.

As set out above, in various embodiments, systems according to the present disclosure ingest structured and unstructured patient EMR data. With natural language processing, a detailed profile of key clinical findings is created for the patient to compare to trial eligibility criteria. Site clinical trials from www.clinicaltrials.gov or other sources are also ingested. The clinical data and criteria for inclusion/exclusion are determined. The clinician may then be provided with a ranked list of relevant trials for a patient as well as the trials from which they were excluded. In some embodiments, clinical trial coordinators are provided with a workbench to help manage and track patients through the recruitment process and to share progress across networks in real time. In some embodiments, the available patient population is continually reviewed for potential eligibility for all trials at the site and the workbench is updated accordingly.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

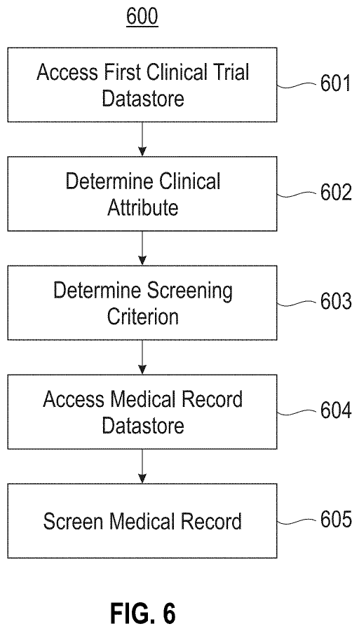

Referring to FIG. 6, a method for detecting cohorts, arms and phases in a clinical study is illustrated. At 601, a first datastore is accessed. The first datastore comprises a clinical trial description. The clinical trial description includes a reference to a cohort, arm, or phase of a clinical study. The clinical trial description is analyzed to identify an entity type. The entity type is a cohort, arm or phase reference. At 602, a clinical attribute associated with the entity type is determined within the clinical trial description. At 603, a screening criterion associated with the clinical attribute is determined. At 604, a second datastore is accessed. The second datastore comprises a medical record of a patient. At 605, the medical record is screened against the screening criterion.

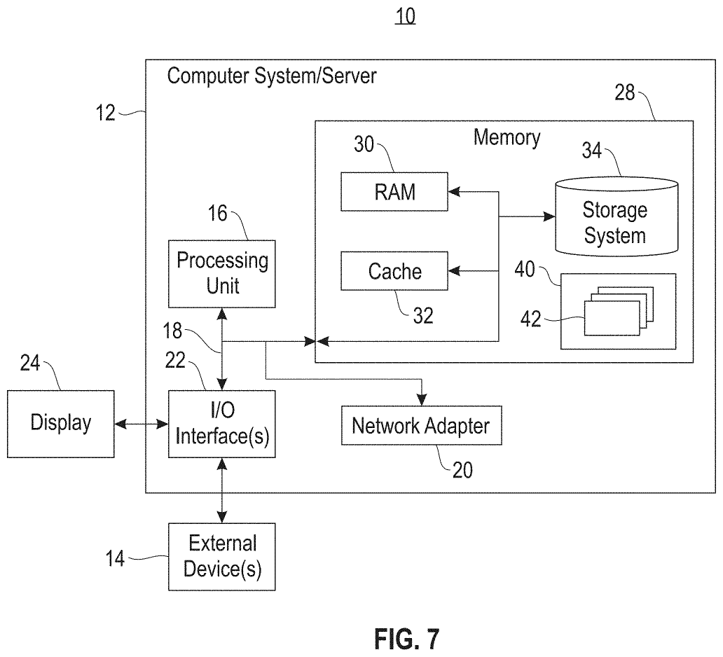

Referring now to FIG. 7, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 7, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of detecting cohorts, arms and phases in a clinical study comprising:
accessing a first datastore, the first datastore comprising a clinical trial description, the clinical trial description including a reference to a cohort, arm, or phase of a clinical study;
analyzing the clinical trial description to identify an entity type, the entity type being a cohort, arm or phase reference;
determining a clinical attribute associated with the entity type within the clinical trial description;
determining a screening criterion associated with the clinical attribute;
accessing a second datastore, the second datastore comprising a medical record of a patient;
screening the medical record against the screening criterion.

2. The method of claim 1, wherein analyzing the clinical trial description comprises:
extracting a cohort, arm or phase by Natural Language Processing.

3. The method of claim 1, wherein analyzing the clinical trial description comprises:
mapping the entity type to a definition of the detected entity type and the section of the clinical trial description wherein the entity was detected.

4. The method of claim 1, wherein analyzing the clinical trial description comprises:
evaluating structural relationships within the clinical trial description.

5. The method of claim 1, wherein analyzing the clinical trial description comprises:
inspecting metadata of the clinical trial description for indications of the entity type.

6. The method of claim 1, wherein analyzing the clinical trial description comprises:
determining disease characteristics associated with the entity type.

7. The method of claim 1, further comprising:
normalizing the cohort, arm, or phase entity names within the clinical trial description.

8. The method of claim 7, wherein normalizing comprises:
extracting a cohort, arm or phase with NLP techniques such that the extracted data is free of modification or merging with other explicit names.

9. The method of claim 7, wherein normalizing comprises:
referencing patient characteristic requirements for a criterion to apply.

10. The method of claim 7, wherein normalizing comprises:
employing anaphora to identify a cohort, arm or phase name based on prior trial information.

11. The method of claim 7, wherein normalizing comprises:
clustering to reduce redundancy across cohort, arm & phase names by selecting a single descriptor to identify a plurality of groups with overlapping content.

12. A system comprising:
a first datastore comprising a clinical trial description;
a second datastore comprising a medical record of a patient;
a processor;
a computer readable storage medium having program instructions embodied therewith, the program instructions executable by the processor to cause the processor to perform a method comprising:
accessing the clinical trial description from the first datastore, the clinical trial description including a reference to a cohort, arm, or phase of a clinical study;
analyzing the clinical trial description to identify an entity type, the entity type being a cohort, arm or phase reference;
determining a clinical attribute associated with the entity type within the clinical trial description;
determining a screening criterion associated with the clinical attribute;
accessing the medical record of a patient from the second datastore;
screening the medical record against the screening criterion.

13. A computer program product for detecting cohorts, arms and phases in clinical studies, the computer program product comprising a computer readable storage medium having program instructions embodied therewith, the program instructions executable by a processor to cause the processor to perform a method comprising:
accessing a first datastore, the first datastore comprising a clinical trial description, the clinical trial description including a reference to a cohort, arm, or phase of a clinical study;
analyzing the clinical trial description to identify an entity type, the entity type being a cohort, arm or phase reference;
determining a clinical attribute associated with the entity type within the clinical trial description;
determining a screening criterion associated with the clinical attribute;
accessing a second datastore, the second datastore comprising a medical record of a patient;
screening the medical record against the screening criterion.

14. The computer program product of claim 13, wherein analyzing the clinical trial description comprises:
extracting a cohort, arm or phase by Natural Language Processing.

15. The computer program product of claim 13, wherein analyzing the clinical trial description comprises:
mapping the entity type to a definition of the detected entity type and the section of the clinical trial description wherein the entity was detected.

16. The computer program product of claim 13, wherein analyzing the clinical trial description comprises:
evaluating structural relationships within the clinical trial description.

17. The computer program product of claim 13, wherein analyzing the clinical trial description comprises:
inspecting metadata of the clinical trial description for indications of the entity type.

18. The computer program product of claim 13, wherein analyzing the clinical trial description comprises:
determining disease characteristics associated with the entity type.

19. The computer program product of claim 13, the method further comprising:
normalizing the cohort, arm, or phase entity names within the clinical trial description.

20. The computer program product of claim 19, wherein normalizing comprises:
extracting a cohort, arm or phase with NLP techniques such that the extracted data is free of modification or merging with other explicit names.
